(19) United States
(12) Patent Application Publication
(10) Pub. No.: US 2008/0125846 A1
(43) Pub. Date: May 29, 2008

(54) METHOD AND SYSTEM FOR STENT PLACEMENT

(75) Inventors: Vianney Pierre Battle, Salt Lake City, UT (US); Richard Auguste Leparmentier, Salt Lake City, UT (US)

Correspondence Address: PETER VOGEL;GE HEALTHCARE, 3000 N. GRANDVIEW BLVD., SN-477, WAUKESHA, WI, 53188, US

(73) Assignee: GENERAL ELECTRIC COMPANY, Schenectady, NY (US)

(21) Appl. No.: 11/564,320

(22) Filed: Nov. 29, 2006

(51) Int. Cl. A61F 2/84

(52) U.S. Cl. 623/1.11

(57) ABSTRACT

A method for placing a stent is disclosed herein. The method includes obtaining a first pre-acquired image of a patient taken at a first orientation, and obtaining a second pre-acquired image of the patient taken at a second orientation. The method also includes navigating a catheter toward a predetermined location after obtaining the first pre-acquired image and the second pre-acquired image. Navigating a catheter includes estimating the position of the catheter, and conveying the estimated position of the catheter by superimposing a graphical representation of the catheter onto the first pre-acquired image and the second pre-acquired image. The method for placing a stent also includes releasing the stent from the catheter after the catheter reaches the predetermined location. A corresponding system for placing a stent is also provided.

### FIELD OF THE INVENTION

This disclosure relates generally to a method and a system for placing a stent.

### BACKGROUND OF THE INVENTION

A stent is a metal coil or mesh tube that can be placed within a lumen, which is typically a blood vessel, in order to provide support and/or to keep the lumen open. Stents may be implemented to treat a variety of medical conditions such, for example, an aneurysm which is the dilation of a blood vessel resulting in stretching of the vessel wall, or a stenosis which is a partial occlusion of a blood vessel.

A conventional procedure for placing a stent includes the following sequence of steps. A guidewire is initially inserted at the point of entry, which is usually a small percutaneous incision in the arm or groin, and is then transferred through one or more blood vessels to the target site (e.g., a site defined at or near the aneurysm or the stenosis). Thereafter a hollow generally cylindrical catheter is slipped over the guidewire and directed to the target site by following the guidewire. The stent can be compressed or compacted in order to facilitate its navigation through the body, and is preferably transferred through the catheter to the target site in its compressed state. Thereafter, the stent is expanded to support a localized region of the vessel wall and/or to keep the vessel open.

The stent must be precisely positioned at a predetermined location within the blood vessel (e.g., at the dilation or occlusion) in order to most effectively treat the underlying medical condition. Stent placement precision is related to the accuracy with which the guidewire and catheter locate the target site. It is therefore known to implement surgical navigation in order to more accurately direct the guide wire and/or the catheter to the target site.

Surgical navigation may be based on any known tracking technology such as, for example, electromagnetic tracking technology. The surgical navigation system determines the position and/or orientation of a medical device (e.g., a guidewire or a catheter) and conveys this location to a user. The position and orientation information can be conveyed by virtually superimposing a graphic representation of the distal end of the medical device onto a patient image. The patient image is generally acquired using a conventional C-arm fluoroscopy device. The fluoroscopy device takes approximately 30 images per second so the medical device can be viewed in real-time or near real-time as it passes through the patient. Accordingly, the user receives visual feedback to help navigate or guide the medical device to the target site.

Although the amount of radiation used for making the fluoroscopic images is small, it is generally desirable to limit radiation exposure as much as possible while still being able to accurately navigate the medical device.

### BRIEF DESCRIPTION OF THE INVENTION

The above-mentioned shortcomings, disadvantages and problems are addressed herein which will be understood by reading and understanding the following specification.

In an embodiment, a method for placing a stent includes obtaining a first pre-acquired image of a patient taken at a first orientation, and obtaining a second pre-acquired image of the patient taken at a second orientation. The method for placing a stent also includes navigating a catheter toward a predetermined location after obtaining the first pre-acquired image and the second pre-acquired image. Navigating a catheter includes estimating the position of the catheter, and conveying the estimated position of the catheter by superimposing a graphical representation of the catheter onto the first pre-acquired image and the second pre-acquired image. The method for placing a stent also includes releasing the stent from the catheter after the catheter reaches the predetermined location.

In another embodiment, a method for placing a stent includes obtaining a three-dimensional image, obtaining a first pre-acquired image of a patient taken at a first orientation, obtaining a second pre-acquired image of the patient taken at a second orientation, and registering the three-dimensional image with the first pre-acquired image and the second pre-acquired image. The method for placing a stent also includes navigating a catheter toward a predetermined location after registering the three-dimensional image. Navigating a catheter includes estimating the position of the catheter, and conveying the estimated position of the catheter by superimposing a graphical representation of the catheter onto the registered three-dimensional image. The method for placing a stent also includes releasing the stent from the catheter after the catheter reaches the predetermined location.

In yet another embodiment, a method for placing a stent includes obtaining a three-dimensional image, deriving stent placement planning information from the three-dimensional image, obtaining a first pre-acquired image of a patient taken at a first orientation, obtaining a second pre-acquired image of the patient taken at a second orientation, and registering the three-dimensional image with the first pre-acquired image and the second pre-acquired image. The method for placing a stent also includes navigating a catheter toward a predetermined location after registering the three-dimensional image. Navigating a catheter includes estimating the position of the catheter, and conveying the estimated position of the catheter by superimposing a graphical representation of the catheter onto the registered three-dimensional image. The method for placing a stent also includes releasing the stent from the catheter after the catheter reaches the predetermined location.

In yet another embodiment, a system for placing a stent includes a computer, and an imaging device operatively connected to the computer. The imaging device is adapted to obtain a first pre-acquired image taken at a first orientation, and a second pre-acquired image taken at a second orientation. The system for placing a stent also includes a position detection process in communication with the computer. The position detection process is adapted to estimate the position of a catheter. The catheter is adapted to selectively deploy the stent. The system for placing a stent also includes a display operatively connected to the computer. The display is configured to convey the estimated position of the catheter by superimposing a graphical representation of the catheter onto the first pre-acquired image and the second pre-acquired image. Feedback from the display conveying the estimated position of the catheter may be implemented to guide the catheter to a predetermined location at which the stent is deployed.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram of a navigation system in accordance with an embodiment;

FIG. 2 is a block diagram illustrating a method in accordance with an embodiment;

FIG. 3 is a block diagram illustrating a method in accordance with another embodiment; and

FIG. 4 is a block diagram illustrating a method in accordance with another embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

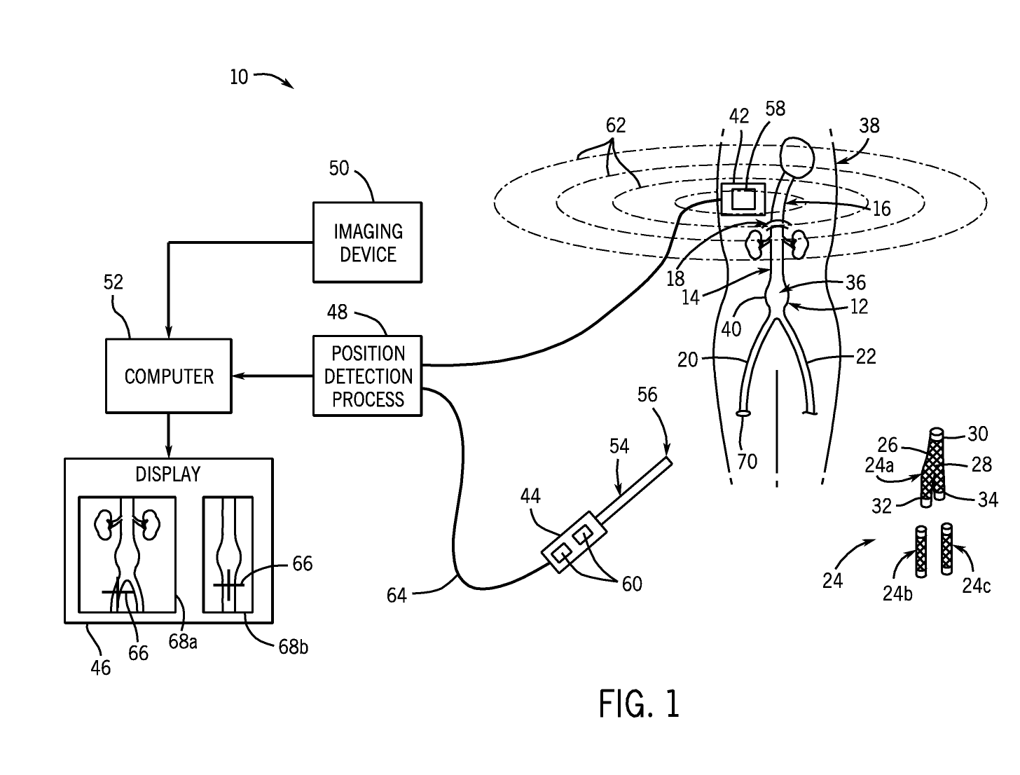

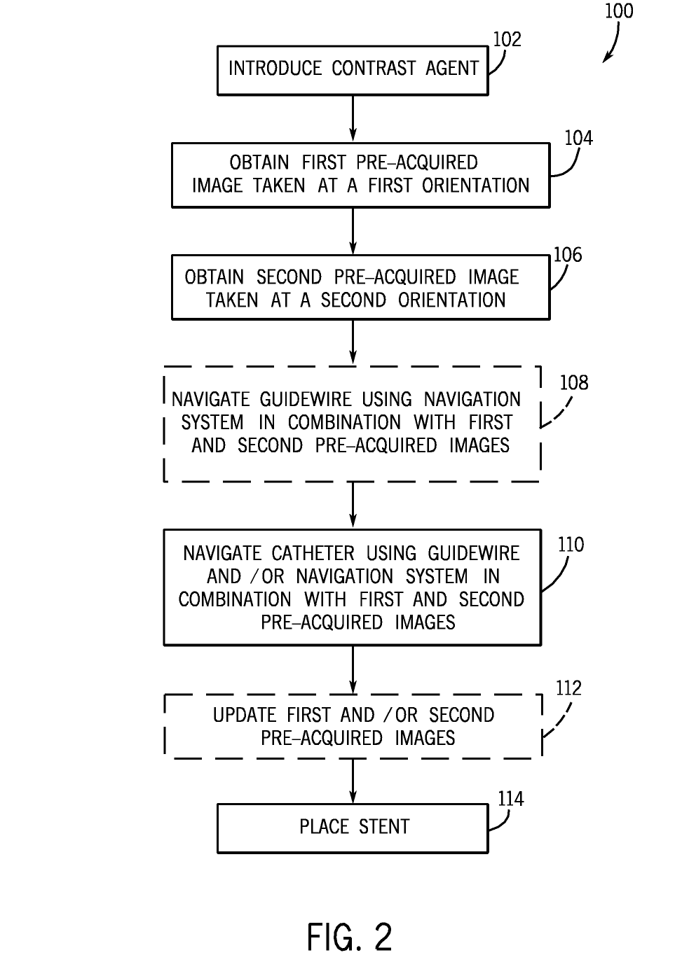

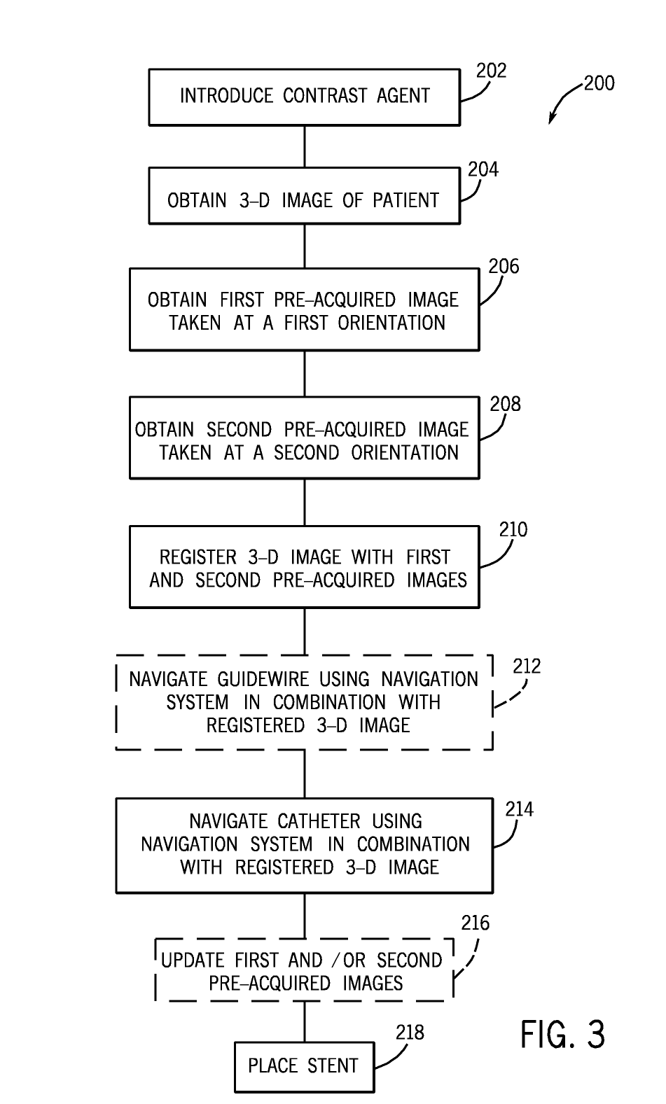

Referring to FIG. 1, an exemplary navigation system 10 is shown. The navigation system 10 and the subsequently described methods 100, 200 (shown in FIGS. 2 and 3, respectively) will be described as being applied to treat an abdominal aortic aneurysm (AAA) 12 for exemplary purposes. It should, however, be appreciated that the navigation system 10 and the methods 100, 200 may also be implemented to treat other types of aneurysms and other medical conditions.

An AAA is a specific type of aneurysm that occurs in the abdominal aorta 14, which is the portion of the aorta 16 generally defined between the diaphragm 18 and the iliac vessels 20, 22. A stent 24 has been developed specifically for the treatment of an AAA. The stent 24 generally includes three components 24a, 24b and 24c.

The stent components 24a, 24b and 24c each include a wire mesh frame 26 that is selectively compressible and expandable. A sleeve 28 is attached, such as with adhesive, to the wire mesh frame 26 so that these two components compress and expand together. When expanded, the wire mesh frame 26 and the attached sleeve 28 form a tubular structure through which fluid is transferable. According to one embodiment, the wire mesh frame 26 is comprised of an alloy, and the sleeve 28 is comprised of a thin plastic material.

The first stent component 24a is generally Y-shaped defining a body 30 and a pair of legs 32, 34 extending therefrom. The second and third stent components 24b, 24c are generally cylindrical and are each adapted for attachment to one of the legs 32, 34. The first stent component 24a is placed in the abdominal aorta 14 near the iliac vessels 20, 22. The second stent component 24b is placed in the iliac vessel 20, and is thereafter attached to the leg 32 of the first stent component 24a. Similarly, the third stent component 24c is placed in the iliac vessel 22, and is thereafter attached to the leg 34 of the first stent component 24a.

The stent components 24a, 24b and 24c are generally transferred to the target site 36 as compressed members in order to facilitate their transmission through the patient 38. Thereafter, the stent components 24a, 24b and 24c are expanded to support a localized region of the vessel wall 40. When expanded, the tubular geometry of the stent components 24a, 24b and 24c facilitates the transfer of blood therethrough. By positioning the stent components 24a, 24b and 24c within the abdominal aorta 14, the iliac vessel 20 and the iliac vessel 22, respectively, blood is directed through the stent 24 without contacting the dilated vessel wall 40 forming the aneurysm 12. Therefore, the pressure generated by the patient's circulatory system is prevented from reaching the dilated vessel wall 40 by locally containing such pressure within the stent components 24a, 24b and 24c. Alleviating the pressure applied to the dilated vessel wall 40 in the manner described greatly diminishes the risks associated with the aneurysm 12.

The navigation system 10 includes a reference unit 42, a remote unit 44, a display 46, a position detection process 48, an imaging device 50 and a computer 52. The reference unit 42 can be rigidly attached to the patient 38 near the target site 36 in a conventional manner. A reference unit attached in this manner is also referred to as a “dynamic reference” because it moves along with the patient. The remote unit 44 is attached to a medical device 54. The medical device 54 will be described as a catheter for exemplary purposes, however, other medical devices and surgical instruments may also be implemented. The present invention will hereinafter be described in accordance with an embodiment wherein the reference unit 42 includes a field generator 58, and the remote unit 44 includes one or more field sensors 60. It should, however, be appreciated that according to alternate embodiments the reference unit may include the field sensors and the remote unit may include the field generator.

The field generator 58 in the reference unit 42 generates a position characteristic field 62 in an area that includes the target site 36. The field sensors 60 in the remote unit 44 produce sensor signals (not shown) in response to the sensed position characteristic field 62. The sensor signals are transmitted or input into the position detection process 48. The sensor signals may be transmitted via communication line 64, or may be wirelessly transmitted. The position detection process 48 is adapted to determine the location of the remote unit 44 relative to the reference unit 42. A known calibration procedure can be implemented to estimate the location of the distal end or tip 56 of the medical device 54.

The location of the medical device 54 may be conveyed via the display 46. According to a preferred embodiment, a graphical representation 66 of the distal end 56 is virtually superimposed onto one or more patient images 68a, 68b. More precisely, the graphical representation 66 of the distal end 56 is virtually superimposed onto the portion of the images 68a, 68b that corresponds to the actual location of the distal end 56 within the patient 38. The images 68a, 68b may, for example, represent different views (e.g., a front-to-back or anterior-posterior (AP) view and a side or lateral view) of the patient 38. The graphical representation 66 may include a dot or cross hairs identifying just the distal end 56, or may include a more complete rendering showing the medical device 54 in detail. According to one embodiment, the patient images 68a, 68b are obtained using the imaging device 50 which will hereinafter be described as being a C-arm fluoroscope in accordance with an exemplary embodiment. It should, however, be appreciated that other known imaging devices may also be implemented.

Referring to FIG. 2, a block diagram illustrates a method 100 for placing the stent 24 (shown in FIG. 1). The individual blocks shown in FIG. 2 represent steps that may be performed in accordance with the method 100.

Referring now to FIGS. 1 and 2, at step 102 a contrast agent is introduced such as by injection into the patient 38. This step is preferably implemented because conventional x-ray imaging technology shows the skeletal structure but does not show blood vessels clearly. Therefore, by implementing contrast agent in combination with an x-ray device such as a fluoroscope, a detailed image of the patient's vascular system can be obtained.

At step 104, a first pre-acquired image 68a of the patient 38 is obtained at a first orientation. At step 106, a second pre-acquired image 68b of the patient 38 is obtained at a second orientation. For purposes of the present disclosure, a “pre-acquired” image is an image taken before the medical device 54 is navigated or guided toward the target site 36 using feedback from the navigation system 10. The first and second pre-acquired images 68a, 68b can be taken with the imaging device 50, which is generally a C-arm fluoroscopic imaging device. The first orientation may, for example, include a front-to-back or AP orientation, and the second orientation may include a side or lateral orientation.

Step 108 is an optional step wherein a guidewire (not shown) is inserted at the point of entry 70, which is usually a small percutaneous incision in the groin, and is then navigated through the iliac vessel 20 to a predetermined location within the target site 36 using the navigation system 10 in combination with the first and second pre-acquired images 68a, 68b. The first and second pre-acquired images 68a, 68b are preferably simultaneously shown on the display 46, and a graphical representation 66 of the guidewire is virtually superimposed onto the portion of the pre-acquired images 68a, 68b that corresponds to the actual location of the guidewire within the patient 38. Therefore, the actual position and orientation of the guidewire relative to the patient 38 can be visually conveyed in order to help navigate the guidewire to the target site 36.

Advantageously, the simultaneous depiction of multiple images taken at different orientations allows the actual position of the guidewire to be more clearly conveyed in three-dimensions. While it may be known to show a graphical representation of a medical device superimposed on sequentially displayed images taken at different orientations, it has not been possible to represent the multiple images simultaneously for the purpose of placing a stent. This is because conventional navigation systems are generally configured to display a first real-time image taken at a first C-arm position, then the C-arm is moved to a second position at which a second real-time image is taken and displayed. As the C-arm cannot be in more than one place at a time, conventional navigation systems do not display more than one image at a time.

It should be appreciated that radiation exposure can be reduced by navigating the guidewire in the manner previously described with respect to step 108. During a conventional surgically navigated procedure, x-ray images may be taken at a rate of 20 per second throughout the course of the entire procedure. By superimposing the graphical representation of the guidewire onto pre-acquired still images (i.e., the first image 68a of step 104 and the second image 68b of step 106), radiation exposure is potentially limited to that which is necessary to take only two images.

It should also be appreciated that navigating the guidewire in the manner previously described with respect to step 108 is potentially more efficient. In order to show multiple images during a conventional surgically navigated procedure, a C-arm x-ray device is rotated back and forth between multiple positions. Therefore, it may have previously been necessary for a user to wait before advancing a medical device until the C-arm was rotated into position and the appropriate image was displayed. By navigating the guidewire in the manner described hereinabove with respect to step 108, there is never a need to wait for an image because all the images can be simultaneously and continuously displayed throughout the course of the procedure.

At step 110 a catheter 54 is inserted at the point of entry 70, and is then navigated through the iliac vessel 20 to a predetermined location within the target site 36 using a guidewire (not shown) and/or the navigation system 10 in combination with the first and second pre-acquired images 68a, 68b. The first and second pre-acquired images 68a, 68b are preferably simultaneously shown on the display 46, and a graphical representation 66 of the catheter 54 is virtually superimposed onto the portion of the images 68a, 68b that corresponds to the actual location of the catheter 54 within the patient 38. Therefore, the actual position and orientation of the catheter 54 relative to the patient 38 can be visually conveyed in order to help navigate the catheter 54 to the target site 36. Advantageously, this simultaneous depiction of multiple images 68a, 68b taken at different orientations allows the actual position and orientation of the catheter 54 to be conveyed in three-dimensions. The previously described advantages associated with navigating a guidewire in accordance with step 108 are also applicable to the navigation of a catheter in accordance with step 110. If the optional step 108 was performed, the catheter 54 may additionally or alternatively be navigated to the target site 36 using the guidewire in a conventional manner.

Step 112 is an optional step wherein the imaging device 50 is implemented to update the first and/or second pre-acquired image 68a, 68b. This step may be performed at any point during the procedure. The first and/or second pre-acquired images 68a, 68b may be updated in accordance with step 112 as frequently as desired. The most recently updated image preferably replaces a corresponding subsequent image on the display 46. Thereafter, navigation proceeds with respect to the most recently updated images in the manner described hereinabove.

At step 114, the stent 24 is placed. The stent 24 is generally disposed in its compressed state within the catheter 54. After visual feedback from the navigation system 10 confirms that the catheter 54 is properly positioned relative to the aneurysm 12, the stent 24 is released from the catheter 54 into the blood vessel 14. Thereafter, the stent 24 is expanded in a conventional manner. If the stent 24 includes multiple components, subsequent stent components may be similarly placed.

According to one embodiment, the navigation system 10 can superimpose a virtual image (not shown) of the deployed stent onto the portion of the pre-acquired images 68a, 68b that corresponds to the actual stent deployment position. This allows a user to see how the stent 24 will look in its fully expanded state within the blood vessel 14 before choosing to actually release the stent 24. Accordingly, the user has access to additional visual feedback to ensure that the stent 24 is precisely placed at the location selected to optimally treat the aneurysm 12.

Referring to FIG. 3, a block diagram illustrates a method 200 for placing the stent 24 (shown in FIG. 1). The individual blocks shown in FIG. 3 represent steps that may be performed in accordance with the method 200.

Referring now to FIGS. 1 and 3, at step 202 a contrast agent is introduced such as by injection into the patient 38 in order to better show the vascular system. At step 204, a three-dimensional image (not shown) of the patient is obtained such as, for example, with a CT device (not shown). At step 206, a first pre-acquired image 68a of the patient 38 is obtained at a first orientation. At step 208, a second pre-acquired image 68b of the patient 38 is obtained at a second orientation. The first and second pre-acquired images 68a, 68b can be taken with the imaging device 50, which is generally a C-arm fluoroscopic imaging device. The first orientation may, for example, include a front-to-back or AP orientation, and the second orientation may include a side or lateral orientation.

At step 210, the three-dimensional image (not shown) is registered with the first and second pre-acquired images 68a, 68b. For purposes of this disclosure, the term “register” refers to the process of aligning or coordinating a plurality of images in order to locate common features. This step is necessary to ensure that the position and orientation data from the navigation system 10 is accurately coordinated with the three-dimensional image. In other words, this step is necessary to ensure that the position and orientation data from the navigation system 10 can be precisely superimposed onto the portion of the three-dimensional image that reflects the position of the medical device 54 within the patient 38.

Step 212 is an optional step wherein a guidewire (not shown) is inserted at the point of entry 70 and is navigated through the iliac vessel 20 to a predetermined location within the target site 36 using the navigation system 10 in combination with the registered three-dimensional image (not shown). The navigation system 10 virtually superimposes a graphical representation 66 of the guidewire onto the portion of the registered three-dimensional image that corresponds to the actual location of the guidewire within the patient 38. Therefore, the actual position and orientation of the guidewire relative to the patient 38 can be visually conveyed in order to help navigate the guidewire to the target site 36.

At step 214 a catheter is inserted at the point of entry 70, and is navigated through the iliac vessel 20 to a predetermined location withing the target site 36 using a guidewire (not shown) and/or the navigation system 10 in combination with the registered three-dimensional image (not shown). The navigation system 10 virtually superimposes a graphical representation 66 of the catheter 54 onto the portion of the registered three-dimensional image that corresponds to the actual location of the catheter 54 within the patient 38. Therefore, the actual position and orientation of the catheter 54 relative to the patient 38 can be visually conveyed in order to help navigate the catheter 54 to the target site 36. If the optional step 212 was performed, the catheter 54 may additionally or alternatively be navigated to the target site 36 using the guidewire in a conventional manner.

As the registered three-dimensional image is also preferably pre-acquired, all of the advantages described hereinabove with respect to step 108 of FIG. 2 also apply to steps 212 and 214. More precisely, steps 212 and 214 convey the position of the medical device (i.e., the guidewire or the catheter) in three-dimensions, they minimize radiation exposure, and they improve efficiency as compared to a conventional navigation procedure.

Step 216 is an optional step wherein the imaging device 50 is implemented to update the first and/or second pre-acquired image 68a, 68b. This step may be performed at any point during the procedure. The first and/or second pre-acquired images 68a, 68b may be updated in accordance with step 216 as frequently as desired. The updated images may replace the registered three-dimensional image (not shown) or may be shown in addition to the registered three-dimensional image on the display 46. Thereafter, navigation can proceed with respect to the updated images and/or the registered three-dimensional image in the manner described hereinabove.

At step 218, the stent 24 is placed. The stent 24 is generally disposed in its compressed state within the catheter 54. After visual feedback from the navigation system 10 confirms that the catheter 54 is properly positioned relative to the aneurysm 12, the stent 24 is released from the catheter 54 into the blood vessel 14. Thereafter, the stent 24 is expanded in a conventional manner. If the stent 24 includes multiple components, subsequent stent components may be similarly placed.

According to one embodiment, the navigation system 10 can superimpose a virtual image (not shown) of the deployed stent onto the portion of the registered three-dimensional image (not shown) that corresponds to the actual stent deployment position. This allows a user to see how the stent 24 will look in its fully expanded state within the blood vessel 14 before choosing to actually release the stent 24. Accordingly, the user has access to additional visual feedback to ensure that the stent 24 is precisely placed at the location selected to optimally treat the aneurysm 12.

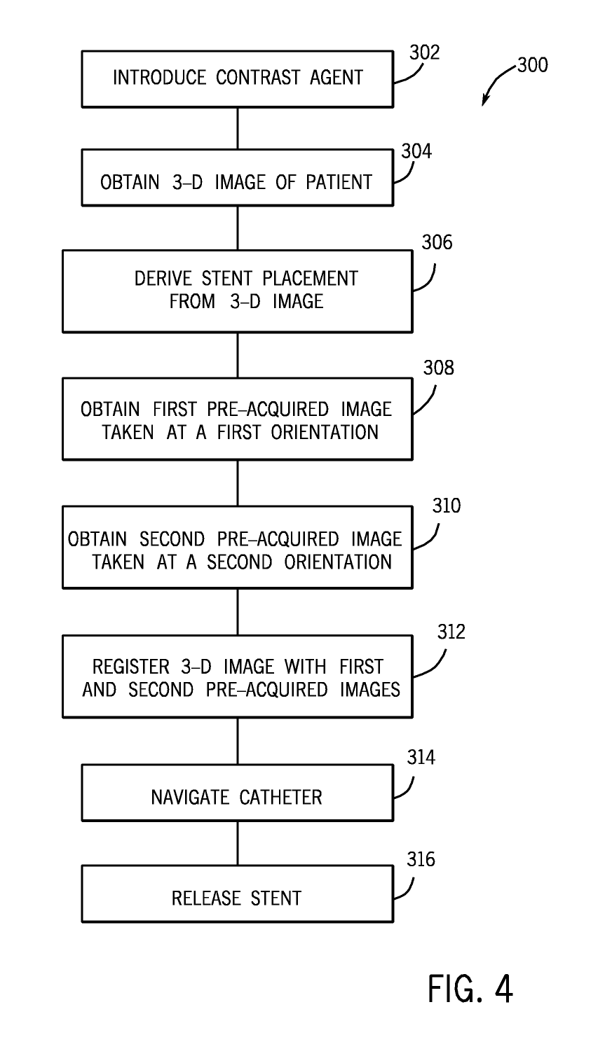

Referring to FIG. 4, a block diagram illustrates a method 300 for placing the stent 24 (shown in FIG. 1). The individual blocks shown in FIG. 4 represent steps that may be performed in accordance with the method 300.

Referring now to FIGS. 1 and 4, at step 302 a contrast agent is introduced such as by injection into the patient 38 in order to better show the vascular system. At step 304, a three-dimensional image (not shown) of the patient is obtained such as, for example, with a CT device (not shown). At step 306, stent placement planning information is derived from the three-dimensional image of step 304. This stent placement planning information will augment the three-dimensional image as it relates to the stent placement.

At step 308, a first pre-acquired image 68a of the patient 38 is obtained at a first orientation. At step 310, a second pre-acquired image 68b of the patient 38 is obtained at a second orientation. The first and second pre-acquired images 68a, 68b can be taken with the imaging device 50, which is generally a C-arm fluoroscopic imaging device. The first orientation may, for example, include a front-to-back or AP orientation, and the second orientation may include a side or lateral orientation.

At step 312, the three-dimensional image (not shown) is registered with the first and second pre-acquired images 68a, 68b. This step is necessary to ensure that the position and orientation data from the navigation system 10 is accurately coordinated with the three-dimensional image. In other words, this step is necessary to ensure that the position and orientation data from the navigation system 10 can be precisely superimposed onto the portion of the three-dimensional image that reflects the position of the medical device 54 within the patient 38.

At step 314, the catheter 54 is navigated toward a predetermined location after registering the three-dimensional image (at step 312) and obtaining the relevant planning information (at step 306). The process of navigating the catheter 54 in accordance with step 314 includes estimating the position of the catheter 54, and conveying the estimated position of the catheter 54 by superimposing a graphical representation of the catheter 54 onto the registered three-dimensional image (not shown) and the relevant planning information (not shown). At step 316, the stent 24 is released from the catheter 54 after the catheter 54 reaches the predetermined location.

According to one embodiment, the navigation system 10 can superimpose a virtual image (not shown) of the deployed stent onto the registered three-dimensional image (not shown) and the relevant planning information (not shown). This allows a user to see how the stent 24 will look in its fully expanded state within the blood vessel 14 before choosing to actually release the stent 24. Accordingly, the user has access to additional visual feedback to ensure that the stent 24 is precisely placed at the location selected to optimally treat the aneurysm 12.

While the invention has been described with reference to preferred embodiments, those skilled in the art will appreciate that certain substitutions, alterations and omissions may be made to the embodiments without departing from the spirit of the invention. Accordingly, the foregoing description is meant to be exemplary only, and should not limit the scope of the invention as set forth in the following claims.

I claim:

1. A method for placing a stent comprising:
obtaining a first pre-acquired image of a patient taken at a first orientation;
obtaining a second pre-acquired image of the patient taken at a second orientation;
navigating a catheter toward a predetermined location after said obtaining the first pre-acquired image and the second pre-acquired image, said navigating a catheter including:
estimating the position of the catheter; and
conveying the estimated position of the catheter by superimposing a graphical representation of the catheter onto the first pre-acquired image and the second pre-acquired image; and
releasing the stent from the catheter after the catheter reaches the predetermined location.

2. The method of claim 1, further comprising introducing contrast agent into the patient before said obtaining the first pre-acquired image or the second pre-acquired image.

3. The method of claim 1, wherein said obtaining a first pre-acquired image includes obtaining a first pre-acquired fluoroscopic image, and said obtaining a second pre-acquired image includes obtaining a second pre-acquired fluoroscopic image.

4. The method of claim 1, wherein one of said first orientation and said second orientation is an anterior-posterior orientation, and the other of said first orientation and said second orientation is a lateral orientation.

5. The method of claim 1, wherein said navigating a catheter toward a predetermined location includes simultaneously displaying the first pre-acquired image and the second pre-acquired image.

6. The method of claim 1, further comprising expanding the stent after said releasing the stent from the catheter.

7. The method of claim 1, further comprising superimposing a graphical representation of the deployed stent onto the first pre-acquired image and/or the second pre-acquired image before said releasing the stent from the catheter.

8. The method of claim 1, further comprising navigating a guidewire toward a second predetermined location after said obtaining the second pre-acquired image and before said navigating a catheter, said navigating a guidewire including:
estimating the position of the guidewire; and
conveying the estimated position of the guidewire by superimposing a graphical representation of the guidewire onto the first pre-acquired image and the second pre-acquired image.

9. The method of claim 1, further comprising updating the first pre-acquired image and/or the second pre-acquired image.

10. A method for placing a stent comprising:
obtaining a three-dimensional image;
obtaining a first pre-acquired image of a patient taken at a first orientation;
obtaining a second pre-acquired image of the patient taken at a second orientation;
registering the three-dimensional image with the first pre-acquired image and the second pre-acquired image;
navigating a catheter toward a predetermined location after said registering the three-dimensional image, said navigating a catheter including:
estimating the position of the catheter; and
conveying the estimated position of the catheter by superimposing a graphical representation of the catheter onto the registered three-dimensional image; and
releasing the stent from the catheter after the catheter reaches the predetermined location.

11. The method of claim 10, further comprising introducing contrast agent into the patient before said obtaining the three-dimensional image, the first pre-acquired image or the second pre-acquired image.

12. The method of claim 10, wherein said obtaining a first pre-acquired image includes obtaining a first pre-acquired fluoroscopic image, and said obtaining a second pre-acquired image includes obtaining a second pre-acquired fluoroscopic image.

13. The method of claim 10, further comprising expanding the stent after said releasing the stent from the catheter.

14. The method of claim 10, further comprising superimposing a graphical representation of the deployed stent onto the registered three-dimensional image before said releasing the stent from the catheter.

15. The method of claim 10, further comprising navigating a guidewire toward a second predetermined location after said registering the three-dimensional image and before said navigating a catheter, said navigating a guidewire including:
estimating the position of the guidewire; and
conveying the estimated position of the guidewire by superimposing a graphical representation of the guidewire onto the registered three-dimensional image.

16. A method for placing a stent comprising:
obtaining a three-dimensional image;
deriving stent placement planning information from said three-dimensional image;
obtaining a first pre-acquired image of a patient taken at a first orientation;
obtaining a second pre-acquired image of the patient taken at a second orientation;
registering the three-dimensional image with the first pre-acquired image and the second pre-acquired image;
navigating a catheter toward a predetermined location after said registering the three-dimensional image, said navigating a catheter including:
estimating the position of the catheter; and
conveying the estimated position of the catheter by superimposing a graphical representation of the catheter onto the registered three-dimensional image; and
releasing the stent from the catheter after the catheter reaches the predetermined location.

17. The method of claim 16, further comprising introducing contrast agent into the patient before said obtaining the three dimensional image, the first pre-acquired image or the second pre-acquired image.

18. The method of claim 16, wherein said obtaining a first pre-acquired image includes obtaining a first pre-acquired fluoroscopic image, and said obtaining a second pre-acquired image includes obtaining a second pre-acquired fluoroscopic image.

19. The method of claim 16, further comprising expanding the stent after said releasing the stent from the catheter.

20. The method of claim 16, further comprising superimposing a graphical representation of the deployed stent onto the registered three-dimensional image and the stent placement planning information before said releasing the stent from the catheter.

21. The method of claim 16, further comprising navigating a guidewire toward a second predetermined location after said registering the three-dimensional image and before said navigating a catheter, said navigating a guidewire including:
estimating the position of the guidewire; and
conveying the estimated position of the guidewire by superimposing a graphical representation of the guidewire onto the registered three-dimensional image.

22. An system for placing a stent comprising:
a computer;
an imaging device operatively connected to the computer, said imaging device adapted to obtain a first pre-acquired image taken at a first orientation, and a second pre-acquired image-taken at a second orientation;
a position detection process in communication with the computer, said position detection process adapted to estimate the position of a catheter, said catheter being adapted to selectively deploy the stent; and
a display operatively connected to the computer, said display configured to convey the estimated position of the catheter by superimposing a graphical representation of the catheter onto the first pre-acquired image and the second pre-acquired image;
wherein feedback from the display conveying the estimated position of the catheter may be implemented to guide the catheter to a predetermined location at which the stent is deployed.

23. The system of claim 22, wherein said display is configured to simultaneously show the first pre-acquired image and the second pre-acquired image.

24. The system of claim 22, wherein said display is selectively configured to superimpose a graphical representation of the deployed stent onto the first pre-acquired image and/or the second pre-acquired image.

25. The system of claim 22, wherein said imaging device includes a C-arm fluoroscopic imaging device.

26. The system of claim 22, wherein one of said first orientation and said second orientation is an anterior-posterior orientation, and the other of said first orientation and said second orientation is a lateral orientation.
